(19) United States
(12) Patent Application Publication
(10) Pub. No.: US 2026/0356782 A1
(43) Pub. Date: Nov. 12, 2026

(54) PROCESS FOR THE MANUFACTURE OF ISONONANOL FROM RENEWABLY-SOURCED ETHANOL

(71) Applicant: BASF SE, Ludwigshafen am Rhein (DE)

(72) Inventors: Stefan WILLERSINN, Houston, TX (US); Alois KINDLER, Ludwigshafen am Rhein (DE); Christian WEINEL, Ludwigshafen am Rhein (DE); Daniel KECK, Ludwigshafen am Rhein (DE); Thomas MACKEWITZ, Ludwigshafen am Rhein (DE); Dieter RAHN, Ludwigshafen am Rhein (DE); Johannes Lazaros Friedrich ELLER, Ludwigshafen am Rhein (DE)

(21) Appl. No.: 19/123,966

(22) PCT Filed: Oct. 27, 2023
(86) PCT No.: PCT/EP2023/080096

§ 371 Date: Apr. 24, 2025

(30) Foreign Application Priority Data
Oct. 28, 2022 (EP) 22204408.3

(51) Int. Cl. C07C 31/125; C07C 1/24; C07C 2/06; C07C 5/22; C07C 11/02; C07C 29/14; C07C 45/50; C07C 47/02; C07C 67/08; C07C 69/44; C07C 69/80

(52) U.S. Cl. CPC C07C 31/125; C07C 1/24; C07C 2/06; C07C 5/22; C07C 11/02; C07C 29/14; C07C 45/50; C07C 47/02; C07C 67/08; C07C 69/44; C07C 69/80

(57) ABSTRACT

A process for the manufacture of isononanol, comprises subjecting a feedstock comprising a renewably-sourced ethanol to dehydration to produce a renewably-sourced ethylene stream. The renewably-sourced ethylene stream is subjected to an olefin-interconversion to obtain a renewably-sourced C₄-olefin stream, the renewably-sourced C₄-olefin stream comprising n-butenes, isobutene or a mixture of n-butenes and isobutene. The olefin-interconversion comprises (i) and, where required, (ii), or (ii) and (iii): (i) ethylene dimerization to obtain n-butenes; (ii) isomerization of n-butenes obtained according to (i) to obtain isobutene; (iii) blending n-butenes obtained according to (i) with isobutene obtained according to (ii). The renewably-sourced C₄-olefin stream is subjected to a sequence of chemical conversions to obtain isononanol, comprising: oligomerizing the renewably-sourced C₄-olefin olefin stream to produce dibutenes; subjecting the dibutenes to a hydroformylation reaction with syngas to obtain isononanal; and hydrogenating the isononanal to produce isononanol. The process provides a reaction scheme for renewably-sourced isononanol.

### TECHNICAL BACKGROUND

The present invention relates to a process for the manufacture of isononanol from renewably-sourced ethanol.

Isononanol (INA) is an example of an oxo-alcohol which is in high demand worldwide. INA is used essentially in the production of plasticizers. There is a trend towards ‘green’ products which are naturally sourced or renewably sourced.

Ethylene is a cornerstone of the modern petrochemical industries. Important ethylene derivatives (at the end of their respective chains) include (meth)acrylic acid, (meth)acrylic esters, isononanol, ethylhexanol, and ethylene glycols. One of the problems faced by the manufacture of chemicals and intermediates from ethylene is that the starting raw materials are from fossil fuels, such as natural gas or crude oil, which are non-renewable feedstocks. Steam cracking, which employs petroleum fractions and natural gas liquids as feedstocks, is the dominant method for large-scale ethylene production worldwide.

Lower olefins, such as isobutylene or propylene, are of significant interest for industrial and chemical applications. Isobutylene, also known as isobutene or 2-methylpropene, is a hydrocarbon of significant interest that is widely used as an intermediate in the production of industrially important products, including para-xylene, jet fuel blendstocks, gasoline oxygenates, isooctane, methacrolein, methyl methacrylate, and butyl rubber. Propylene is a hydrocarbon of significant interest that is widely used as an intermediate in the production of acrylic acid. Historically, lower olefins have been obtained through the catalytic or steam cracking of fossil fuel feedstocks.

Applicants have realized that the production of ethylene and ethylene derivatives compounds would benefit from the replacement of at least a part of the carbonaceous raw materials of fossil origin by renewable resources, such as carbonaceous matter derived from biomass. Of particular interest is the ethanol feedstock which is produced from renewable resources. Such renewably-sourced ethanol, also referred to as “bioethanol” or “hydrous fuel alcohol” can be prepared in large quantities from organic waste or biomass via fermentation. The different feedstocks for producing ethanol may be sucrose-containing feedstocks, e.g., sugarcane, starchy materials, e.g., corn, starch, wheat, cassava, lignocellulosic biomass, e.g., switchgrass, and/or agricultural waste. The purification or isolation of bioethanol is frequently carried out by complicated, multistage distillation.

Even after the purification processes, the advantage of bioethanol is frequently decreased by small amounts of impurities which it contains. Bioethanol impurities may include oxygen-containing organics, for example other alcohols such as isopropanol, n-propanol, and isobutanol, and/or aldehydes such as acetaldehyde. Bioethanol impurities may further include sulfur-containing impurities, such as inorganic sulfur compounds dialkyl sulfides, dialkyl sulfoxides, alkyl mercaptans, 3-methylthio-1-propanol, and/or sulfur-containing amino acids.

It would be desirable to integrate renewably-sourced ethanol into existing processes designed for the conversion of fossil-derived ethylene or its intermediates. However, some of the impurities may interfere with the downstream processes which use bioethanol as feedstock and which generate chemical products, especially when some of the downstream steps are catalytic conversions.

If efforts are not made to remove at least some of these impurities, the yield of desired intermediate and final products and efficacy of the overall process may be diminished.

US 2008/0312485 discloses a method for continuously producing propylene by dehydrating ethanol obtained from biomass to obtain ethylene and reacting ethylene with n-butene in a metathesis reaction. The n-butene is made by dimerization of ethylene which is obtained from biomass-derived ethanol [0033] and [0061].

WO 2010/066830 discloses the transformation of bioethanol to ethylene. The bioethanol is produced by fermentation of carbohydrates or from synthesis gas made by gasification of biomass. The ethylene is subsequently dimerized or oligomerized to, e.g., 1-butene and/or 1-hexene. The dimeric or oligomeric alpha-olefins are transformed into internal olefins that are subsequently subjected to metathesis with ethylene.

WO 2011/085223 discloses an integrated process to prepare renewable hydrocarbons. The process includes dehydrating renewable isobutanol to form a mixture of linear butenes and isobutene and dehydrating renewable ethanol to ethylene. Subsequently the butene mixture and the ethylene are reacted to form one or more renewable C₃-C₁₆ olefins.

EP 3 067 340 A discloses a process comprising fermenting a renewable source of carbon for the production of a mixture of alcohols comprising ethanol, isopropanol and 1-butanol; joint dehydration of the alcohols to produce a mixture of olefins comprising chiefly ethylene, propylene and linear butenes, the linear butenes being a mixture of 1-butene and 2-butenes (cis- and trans-isomers), besides water and by-products; removal of water, oxygenated compounds and other by-products from the mixture of olefins, to generate a mixture of olefin comprising chiefly ethylene, propylene and linear butenes; and passing the mixture of olefins through an isomerization bed so that 1-butene is isomerized to 2-butene and subsequently passing the mixture of olefins comprising chiefly ethylene, propylene and 2-butenes through a metathesis bed, for reaction between ethylene and 2-butenes, generating additional propylene.

WO 2009/098268 discloses a process for the dehydration of an alcohol to make an olefin. The alcohol may be ethanol that can be obtained from carbohydrates. For this purpose a stream comprising the ethanol and an inert component is contacted with a catalyst to give ethylene. It is indicated that the ethylene can be used for dimerization to butene and then isomerization to isobutene, dimerization to 1-butene, which is isomerized to 2-butene and further converted by metathesis with ethylene to propylene, or conversion to ethylene oxide and glycol. Experimental details are provided only for ethanol dehydration. A similar process is disclosed in WO 2011/089235.

WO 2009/098269 discloses a process for conversion of ethanol that can be obtained from carbohydrates to propylene. The ethanol is dehydrated to ethylene which is reacted with olefins having four carbon atoms or more to give propylene. WO 2009/098267 discloses a similar process.

WO 2021/067294 discloses a process for simultaneously dehydrating, dimerizing and metathesizing a C₂-C₅ alcohol which can be from biobased processes in one reactor to produce a C₂-C₇ olefin.

WO 2009/070858 discloses an integrated process for the production of ethylene-butylene copolymers. The ethylene is obtained by dehydration of ethanol that is produced by the fermentation of sugars. One method of obtaining 1-butylene used for the polymerization is indicated to be dimerization of ethylene produced by dehydration of ethanol that is produced by the fermentation of sugars. No details as to the dimerization are given.

In embodiments, the invention seeks to advise a reaction scheme that provides renewably-sourced light olefins, such as ethylene, butenes and isobutene, which partially or fully replace the light olefins output from a steam cracker. These light olefins are used as building blocks for producing a variety of chemicals of interest. It is desirable that the renewably-sourced light olefins can be blended or used interchangeably with a fossil-derived intermediate of the same chemical structure without necessitating adjustments in downstream processes. This includes that the starting olefins of all branches of the value chains, which historically have been served by the steam cracker output, can be supplied at the same time on a renewably-sourced basis. In this way, the greenhouse gases footprint and/or the carbon footprint for the production of a chemical of interest is at least reduced.

### DETAILED DESCRIPTION OF THE INVENTION

To this effect, the present invention relates to a process for the manufacture of isononanol, said process comprising the steps of:
a) subjecting a feedstock comprising a renewably-sourced ethanol to dehydration to produce a renewably-sourced ethylene stream;
b) subjecting the renewably-sourced ethylene stream to an olefin-interconversion to obtain a renewably-sourced C₄-olefin stream, the renewably-sourced C₄-olefin stream comprising n-butenes, isobutene or a mixture of n-butenes and isobutene; the olefin-interconversion comprising (i) and, where required, (ii), or (ii) and (iii):
   (i) ethylene dimerization to obtain n-butenes;
   (ii) isomerization of n-butenes obtained according to (i) to obtain isobutene;
   (iii) blending n-butenes obtained according to (i) with isobutene obtained according to (ii); and
c) subjecting the renewably-sourced C₄-olefin stream to a sequence of chemical conversions to obtain isononanol, comprising:
   α) oligomerizing the renewably-sourced C₄-olefin olefin stream to produce dibutenes;
   β) subjecting the dibutenes to a hydroformylation reaction with syngas to obtain isononanal; and
   γ) hydrogenating the isononanal to produce isononanol.

The term “isononanol” is understood to relate to a mixture of isomeric nonyl alcohols such as n-nonanol and single- and/or multi-branched nonanols such as 3,5,5-trimethylhexan-1-ol and methyl octanol.

The process of the invention is preferably a continuous process in the sense that at least the upstream steps of a reaction route leading to a chemical of interest, including at least step a) and any of steps b-(i) through (iii) as far as they are involved in the reaction route, are carried out continuously. In a still more preferred embodiment all steps of a reaction route leading to a chemical of interest are carried out continuously. This does not preclude the presence of buffer volumes between subsequent reaction steps in a reaction route.

The present invention is based on the idea of eliminating impurities that are inherently present in renewably-sourced ethanol during the ethylene manufacturing process itself. Hence, the renewably-sourced ethylene, or the renewably-sourced C₄-olefin stream produced therefrom, can be blended or used interchangeably with a fossil-derived intermediate of the same chemical structure without necessitating adjustments in downstream processes.

It is envisaged that the renewably-sourced olefins involved in the process according to the invention may be blended with complementary olefins from other sources. This can ensure the efficient utilization of downstream processes, e.g., for transitional periods when supply of renewably-sourced olefin is limited. These complementary olefins, including complementary ethylene, complementary isobutene and complementary n-butenes, may be fossil-based, partially renewably-sourced or renewably-sourced by another production route.

Hence in an embodiment, the process comprises:
blending the renewably-sourced ethylene with complementary ethylene prior to step b), the complementary ethylene not being obtained from renewably-sourced ethanol in accordance with step a); and/or
blending the renewably-sourced n-butenes with complementary n-butenes prior to step b-(ii), b-(iii) or c), the complementary n-butenes not being obtained from renewably-sourced n-butenes in accordance with steps a) and b-(i); and/or
blending the renewably-sourced isobutene with complementary isobutene prior to step b-(iii) or c), the complementary isobutene not being obtained from renewably-sourced n-butenes in accordance with steps a), b-(i) and b-(ii).

Examples for complementary ethylenes are ethylenes obtained by steam cracking of fossil based feeds, like naphtha, natural gas or crude oil. Examples for complementary n-butenes are n-butenes obtained by steam cracking of fossil based feeds, like naphtha, natural gas or crude oil. Examples for complementary isobutenes are isobutenes obtained by steam cracking of fossil based feeds, like naphtha, natural gas or crude oil.

In another aspect, the invention also relates to a process for enhancing the environmental sustainability of isononanol by blending or replacing a fossil-derived C₄-olefin stream comprising n-butenes, isobutene or a mixture of n-butenes and isobutene with a renewably-sourced C₄-olefin stream of the same composition to obtain a sustainability-enhanced C₄-olefin stream and subjecting the sustainability-enhanced C₄-olefin stream to a sequence of chemical conversions to obtain isononanol, wherein the renewably-sourced C₄-olefin stream is obtained by
a) subjecting a feedstock comprising a renewably-sourced ethanol to dehydration to produce the renewably-sourced ethylene stream; and
b) subjecting the renewably-sourced ethylene stream to an olefin-interconversion, to obtain the renewably-sourced C₄-olefin stream; the olefin-interconversion comprising (i) and optionally (ii), or (ii) and (iii):
   (i) ethylene-dimerization to obtain n-butenes;
   (ii) isomerization of n-butenes obtained according to (i) to obtain isobutene;
   (iii) blending n-butenes obtained according to (i) with isobutene obtained according to (ii);
      the sequence of chemical conversions comprising:
α) oligomerizing the n the renewably-sourced C₄-olefin olefin stream to produce dibutenes;
β) subjecting the dibutenes to a hydroformylation reaction with syngas to obtain isononanal; and
γ) hydrogenating the isononanal to produce isononanol.

The key advantage of the process according to the present invention is that it can be easily integrated into an existing production site in which one or more chemicals of interest are manufactured based on a fossil feedstock, in particular naphtha. This means that fossil-based ethylene and C₄-olefins can be fully or partially substituted by respective renewably-sourced ethylene and C₄-olefins. Hereby, one obtains a respective chemical of interest, the carbon atoms of which are fully or partially based on a renewable-sourced carbon (so-called “green” carbon).

Further benefits occur from the reduction of carbon dioxide emissions. The chemical conversions involved in a reaction route leading to an individual chemical of interest are usually less than 100% selective. The yield losses manifest themselves in the generation of by-products that vary depending on the type of reaction involved. Oxidation reactions of a substrate to a desired product, for example, are almost invariably accompanied to a certain extent by an over-oxidation of the substrate to form carbon oxides, in particular carbon dioxide. By a full or partial replacement of fossil ethylene and C₄-olefins by their renewably-sourced counterparts, the fossil-based carbon dioxide emissions of the entire production site can be reduced because respective emissions resulting from yield losses along the value chain are at least partially based on green carbon. The resulting carbon dioxide emissions therefore do not contribute to the green house emission of the production site.

In addition, in non-oxidative reactions the various species present may undergo a host of side reactions, which generate color forming species, oligomers, and various decomposition products or the like. These are generally removed during work-up, e.g., by distillation, yielding light boiler and/or high boiler fractions in addition to the desired product. The light boiler or high boiler fractions are conventionally used for their calorific value, i.e. combusted as fuel, or exploited as hydrocarbon source, e.g. as steam cracker feed. It should be appreciated that full or partial replacement of fossil ethylene and C₄-olefins by their renewably-sourced counterparts at the beginning of the processing chain reduces the emission of fossil-based carbon dioxide resulting from the combustion of downstream side-products.

Hence, it is envisaged that direct and indirect benefits are associated with the process of the invention with regard to any chemical of interest that is manufactured via the process according to the present invention.

The expressions “renewable” or “renewably-sourced” in relation to a chemical compound are used synonymously and mean a chemical compound comprising a quantity of renewable carbon, i.e., having a reduced or no carbon content of fossil origin. Renewable carbon entails all carbon sources that avoid or substitute the use of any additional fossil carbon from the geosphere. Renewable carbon can come from the biosphere, atmosphere or technosphere-but not from the geosphere. Thus, the expression “renewable” or “renewably-sourced” includes, in particular, biomass-derived chemical compounds. It also includes compounds derived from waste such as polymer residues, or from waste streams of chemical production processes.

The expression “chemical of interest” collectively refers to any desired compound appearing in a value chain starting out from and including ethylene. Thus, the expression includes any intermediates and final products. In certain cases, a chemical compound can be an intermediate and final product at the same time. For example, isobutene can be the final product of a value chain and yet can be an intermediate when it is further processed, if desired.

All patent and literature documents addressed in the following are incorporated herein by reference in their entirety.

Bioethanol is a preferred form of renewably-sourced ethanol, although the scope of the invention is not limited to the use of bioethanol.

In the present invention, bioethanol refers to the ethanol obtained from a biomass feedstock, such as plant or non-crop feedstock containing a carbon source that is convertible to ethanol, for example by microbial metabolism. Typical carbon source examples are starch, sugars like pentoses or hexoses, such as glucose, fructose, sucrose, xylose, arabinose, or degradation products of plants, hydrolysis products of cellulose or juice of sugar canes, beet and the like containing large amounts of the above components.

Biomass feedstock can originate from several sources. Bioethanol production may be based on food crop feedstocks such as corn and sugar cane, sugarcane bagasse, cassava (first generation biofeedstock).

Another source of biomass feedstock is lignocellulosic materials from agricultural crops (second-generation biofeedstock). Potential feedstocks include agricultural residue by-products such as rice, straw (such as wheat, oat and barley straw), rice husk, and corn stover. Biomass feedstock may also be waste material from the forest products industry (wood waste) and saw dust or produced on purpose as an ethanol crop. Switchgrass and napier grass may be used as on-purpose crops for conversion to ethanol.

The first-generation bioethanol is produced in four basic steps:
(1) Enzymatic saccharification or hydrolysis of starch into sugars
(2) Microbial fermentation of sugars
(3) Purification by distillation to give hydrous ethanol
(4) Dehydration (water removal) to produce anhydrous ethanol

Second-generation feedstocks are considered as renewable and sustainable carbon source. Pretreatment of this feedstock is an essential prerequisite before it is subjected to enzymatic hydrolysis, fermentation, distillation, and dehydration. Pretreatment involves milling and exposure to acid and heat to reduce the size of the plant fibers and hydrolyze a portion of the material to yield fermentable sugars. Saccharification utilizes enzymes to hydrolyze another portion to sugar. Finally, fermentation by bioengineered microorganisms converts the various sugars (pentoses and hexoses) to ethanol. The production of bioethanol is well-known and carried out on an industrial large scale.

Renewably-sourced ethanol can also be obtained from carbon-containing waste materials like waste products from the chemical industry, garbage and sewage sludge. The production of ethanol from waste materials can be done by gasification to syngas and catalytic conversion thereof the ethanol, see for example Recent Advances in Thermo-Chemical Conversion of Biomass, 2015, Pages 213-250, https://doi.org/10.1016/B978-0-444-63289-0.00008-9, and Nat Commun 11, 827 (2020), https://doi.org/10.1038/s41467-020-14672-8.

### Dehydration of Renewably-Sourced Ethanol

As a first step, the invention involves the dehydration of renewably-sourced ethanol. The production of ethylene by catalytic dehydration of ethanol is a well-known process. The reaction is commonly carried out at 300 to 400 °C and moderate pressure in the presence of a catalyst. Catalytic effects are reviewed in Ind & Eng Chem Research, 52, 28, 9505-9514 (2013), Materials 6, 101-115 (2013) and ACS Omega, 2, 4287-4296 (2017). Examples for catalysts are activated alumina or silica, phosphoric acid impregnated on coke, heteropoly acids (HPA salts), silica-alumina, molecular sieves such as zeoliths of the ZSM-5 type or SAPO-11 type, other zeolites or modified zeolites of various molecular structures with zeoliths and HPA salts being preferred.

Ethanol dehydration is, for example described in WO 2009/098268, WO 2010/066830, WO 2009/070858 and the prior art discussed therein, WO 2011/085223 and the prior art discussed therein, U.S. Pat. Nos. 4,234,752, 4,396,789, 4,529,827 and WO 2004/078336.

The ethanol dehydration reaction is in general carried out in the vapor phase in contact with a heterogeneous catalyst bed using either fixed bed or fluidized bed reactors. For fixed bed reactors, the operation can be either isothermal (with external heating system) or adiabatic (in the presence of a heat carrying fluid). The feedstock is vaporized and heated to the desired reaction temperature; the temperature drops as the reaction proceeds in the reactor. Multiple reactor beds are usually used in series to maintain the temperature drop in each bed to a manageable range. The cooled effluent from each bed is further heated to bring it to the desired inlet temperature of the subsequent beds. Moreover, a portion of the water is recirculated along with fresh and unreacted ethanol. The presence of water helps in moderating the temperature decrease in each bed.

Prior to dehydration, the renewably-sourced ethanol feedstock may be sent to a pretreatment section to remove mineral contaminants, which would otherwise be detrimental to the downstream catalytic reaction. The pretreatment may involve contacting the renewably-sourced ethanol feedstock with cation and/or anion exchange resins. After a certain period of operation, the resins may be regenerated by passing a regenerant solution through the resin bed(s) to restore their ion exchange capacity. Two sets of beds are preferably operated in parallel to maintain continuous operation. One set of resin beds is suitably regenerated while the other set is being used for pretreatment.

In the isothermal design, the catalyst is placed inside the tubes of multitubular fixed-bed reactors which arranged vertically and surrounded by a shell (tube and shell design). A heat transfer medium, such as molten salts or oil, is circulated inside the shell to provide the required heat. Baffles may be provided on the shell side to facilitate heat transfer. The cooled heating medium is heated externally and is recirculated. The temperature drop on the process side can be reduced as compared to the adiabatic reactor. A better control on the temperature results in increased selectivity for the ethylene formation and reduction in the amount of undesireable by-products. The temperature is maintained at approximately constant levels within the range of 300° to 350 °C. Ethanol conversion is between 98 and 99%, and the selectivity to ethylene is between 94 and 97 mol %. Because of the rate of coke deposition, the catalyst must be regenerated frequently. Depending on the type of catalyst used, the cycle life is between 3 weeks and 4 months, followed by regeneration, for example for 3 days.

In the adiabatic design, the endothermic heat of reaction is supplied by a preheated inert diluent such as steam. Three fixed-bed reactors may typically be used, with intermediate furnaces to reheat the ethanol/steam mixed feed stream to each reactor. Feeding steam with ethanol results in less coke formation, longer catalyst activity, and higher yields.

A further process is a fluidized-bed process. The fluidized-bed system offers excellent temperature control in the reactor, thereby minimizing by-product formation. The heat distribution rate of the fluidized bed operation approaches isothermal conditions. The endothermic heat of reaction is supplied by the hot recycled silica-alumina catalyst returning from the catalyst regenerator. Thus, external heating of the reactor is not necessary.

After dehydration, the reaction mixture is subjected to a separation step. The general separation scheme consists of quickly cooling the reaction gas, for example in a water quench tower, which separates most of the by-product water and the unreacted ethanol from ethylene and other light components which, for example exit from the top of the quench tower. In one type of separation scheme, the water-washed ethylene stream is immediately caustic-washed, for example in a column, to remove traces of CO₂. The gaseous stream may enter a compressor directly or pass to a surge gas holder first and then to a gas compressor. After compression, the gas is cooled with refrigeration and then passed through an adsorber with, for example activated carbon, to remove traces of heavy components, (e.g., C4s), if they are present. The adsorber is followed by a desiccant drying and dust filtering step before the ethylene product leaves the plant. This separation scheme produces 99%+ purity ethylene. If desired, the ethylene is further purified by caustic washing and desiccant-drying, and fractionated in a low-temperature column to obtain the final product.

Several commercial processes are currently in operation, developed by Braskem, Chematur, British Petroleum (BP), and Axens together with Total and IFPEN. The processes differ, e.g., in their process conditions, catalysts and adopted heat integration scheme. The process by BP (now Technip) is called Hummingbird. In this process, a heteropoly acid is used as catalyst, and the reactor operates at 160 to 270 °C and 1 to 45 bar. The unreacted ethanol in recirculated to the reactor. The process developed by Axens is called Atol. Two fixed bed adiabatic reactors, operating at 400 to 500 °C, are used. Chematur's process operates with four adiabatic tubular reactors. Syndol catalysts, with the main components of Al₂O₃—MgO/SiO₂, are employed in this process that was developed by American Halcon Scientific Design, Inc. in the 1980s. In the Braskem process, the adiabatic reactor feed is diluted with steam to a large extent. In such a process, the reactor operates at 180 to 600 °C, preferably 300 to 500 °C, and at 1.9 to 19.6 bar. An alumina or silica-alumina catalyst is used. The Braskem process is described in more detail in U.S. Pat. No. 4,232,179. A process control in accordance with the Braskem process is particularly preferred.

### Dimerization of Ethylene

The process of the invention involves an ethylene-dimerization to obtain n-butenes in accordance with step b)-(i). Any known method can be used for ethylene dimerization to produce n-butenes. A review on dimerization and oligomerization chemistry and technology is given in Catalysis Today, vol. 14(no. 1), Apr. 10, 1992.

Expediently, step B)-(i) Comprises:
contacting the renewably-sourced ethylene stream with a dimerization catalyst in a dimerization zone;
operating said dimerization zone at conditions effective to produce an effluent consisting essentially of n-butenes, a stream consisting essentially of heavier olefins, and optionally an unconverted ethylene stream; and
fractionating the effluent to recover a stream consisting essentially of n-butenes, a stream consisting essentially of heavier olefins, and an optional ethylene stream.

The dimerization catalyst may be homogeneous or heterogeneous. Typical dimerization catalysts are titanium or nickel compounds activated with alkyl aluminium compounds. In general, the Ti(IV) valency is stabilized by selecting the appropriate ligands, alkyl aluminium compound, the solvent polarity and the Al/Ti ratio. Nickel compounds that can catalyse the selective production of butenes are typically based on cationic nickel salts stabilised with phosphine and activated with alkyl aluminium compounds.

In one embodiment, the oligomerization of ethylene is implemented in the presence of a catalytic system in the liquid phase comprising a nickel compound and an aluminum compound. Such catalytic systems are described in the documents FR 2 443 877 and FR 2794 038. The Dimersol E™ process is based on this technology and leads to the industrial production of olefins.

Thus, in one embodiment, the oligomerization of ethylene is implemented in the presence of a catalytic system comprising:
i) at least one bivalent nickel compound,
ii) at least one hydrocarbyl aluminum dihalide of formula AIRX₂, in which R is a hydrocarbyl radical comprising 1 to 12 carbon atoms, such as alkyl, aryl, aralkyl, alkaryl or cycloalkyl, X is a chlorine or bromine atom, and
iii) optionally a Brønsted organic acid.

As the bivalent nickel compound, nickel carboxylates of general formula (R¹COO)₂Ni are preferably used, where R¹ is an optionally substituted hydrocarbyl radical, for example alkyl, cycloalkyl, alkenyl, aryl, aralkyl, or alkaryl, containing up to 20 carbon atoms, preferably a hydrocarbyl radical of 5 to 20 carbon atoms, preferably 6 to 18 carbon atoms. Suitable bivalent nickel compounds include: chloride, bromide, carboxylates such as octoate, 2-ethylhexanoate, decanoate, oleate, salicylate, hydroxydecanoate, stearate, phenates, naphthenates, and acetyl acetonates. Nickel 2-ethylhexanoate is preferably used.

The hydrocarbyl aluminum dihalide compound corresponds to the formula AIRX₂, in which R is a hydrocarbyl radical comprising 1 to 12 carbon atoms, such as alkyl, aryl, aralkyl, alkaryl or cycloalkyl, and X is a chlorine or bromine atom. As examples of such compounds, it is possible to mention ethylaluminum sesquichloride, dichloroethyl aluminum, dichloroisobutyl aluminum, chlorodiethyl aluminum or mixtures thereof.

According to a preferred method, a Brønsted organic acid is used. The Brønsted acid compound corresponds to the formula HY, where Y is an organic anion, for example carboxylic, sulfonic or phenolic. Halocarboxylic acids of formula R²COOH in which R² is a halogenated alkyl radical are preferred, in particular those that contain at least one alpha-halogen atom of the group —COOH with 2 to 10 carbon atoms in all. Preferably, a haloacetic acid of formula CXₚH₃₋ₚ—COOH is used, in which X is fluorine, chlorine, bromine or iodine, with p being an integer from 1 to 3. By way of example, it is possible to cite the trifluoroacetic, difluoroacetic, fluoroacetic, trichloroacetic, dichloroacetic, and chloroacetic acids. It is also possible to use arylsulfonic, alkylsulfonic, and fluoroalkylsulfonic acids, and picric acid and nitroacetic acid. Trifluoroacetic acid is preferably used.

The three components of the catalytic formula can be mixed in any order. However, it is preferable first to mix the nickel compound with the Brønsted organic acid, and then next to introduce the aluminum compound. The molar ratio of the hydrocarbyl aluminum dihalide to the nickel compound, expressed by the Al/Ni ratio, is 2/1 to 50/1, and preferably 2/1 to 20/1. The molar ratio of the Brønsted acid to the nickel compound is 0.25/1 to 10/1, and preferably 0.25/1 to 5/1.

According to a preferred method, the hydrocarbyl aluminum dihalide can be enriched with an aluminum trihalide, the mixture of the two compounds then corresponding to the formula AIRₙX₃₋ₙ, in which R is a hydrocarbyl radical comprising 1 to 12 carbon atoms, such as alkyl, aryl, aralkyl, alkaryl or cycloalkyl, X is a chlorine or bromine atom, and n is a number between 0 and 1. Suitable mixtures include: dichloroethyl aluminum enriched with aluminum chloride, the mixture having a formula AlEt_{0.9}Cl_{2.1}; dichloroisobutyl aluminum enriched with aluminum chloride, the mixture having a formula AliBu_{0.9}Cl_{2.1}; and dibromoethyl aluminum enriched with aluminum bromide, the mixture having a formula AlEt_{0.9}Br_{2.1}.

The reaction for oligomerization of ethylene can be implemented at a temperature of −20 to 80 °C, preferably 40 to 60 °C, under pressure conditions such that the reagents are kept at least for the most part in the liquid phase or in the condensed phase. The pressure is generally between 0.5 and 5 MPa, preferably between 0.5 MPa and 3.5 MPa. The time of contact is generally between 0.5 and 20 hours, preferably between 1 and 15 hours.

The oligomerization stage can be implemented in a reactor with one or more reaction stages in a series, with the ethylene feedstock and/or the catalytic composition that is preferably pre-conditioned in advance being introduced continuously, either in the first stage, or in the first stage and any other one of the stages. At the outlet of the reactor, the catalyst can be deactivated, for example by injection of ammonia and/or an aqueous solution of soda and/or an aqueous solution of sulfuric acid. The unconverted olefins and alkanes that are optionally present in the feedstock are then separated from the oligomers by a separation stage, for example by distillation or washing cycles by means of caustic soda and/or water.

The conversion per pass is generally 85 to 98%. The selectivity of n-butenes that are formed is generally between 50 and 80%. The n-butenes consist of butene-2 (cis- and trans-) and butene-1.

The effluent generally contains less than 0.2% by weight of isobutene, or even less than 0.1% by weight of isobutene.

### Separation of a Stream Rich in n-Butenes

The effluent that is obtained by dimerization of ethylene may be subjected to a separation stage in such a way as to obtain an n-butene-enriched fraction.

The separation can be carried out by evaporation, distillation, extractive distillation, extraction by solvent or else by a combination of these techniques. These processes are known by one skilled in the art. Preferably, a separation of the effluent that is obtained by oligomerization of ethylene is carried out by distillation.

Preferably, the effluent of the oligomerization is sent into a distillation column system comprising one or more columns that makes it possible to separate, on the one hand, n-butenes from ethylene, which can be returned to the oligomerization reactor, and heavier olefins with 5 carbon atoms and more.

The higher olefins may be subjected to hydrogenation so as to obtain renewably-sourced naphtha. “Renewably-sourced naphtha” shall mean naphtha produced from renewable sources. It is a hydrocarbon composition, consisting of mainly paraffins. The molecular weight of this renewably-sourced naphtha may range from hydrocarbons having 5 to 8 carbon atoms. Renewably-sourced naphtha can be used as a feedstock in steam-cracking to produce renewably-sourced light olefins, dienes and aromatics.

Hence, in an embodiment, step b)-(i) comprises:
contacting the renewably-sourced ethylene stream with a dimerization catalyst in a dimerization zone;
operating said dimerization zone at conditions effective to produce an effluent consisting essentially of n-butenes, a stream consisting essentially of heavier olefins, and optionally an unconverted ethylene stream;
fractionating the effluent to recover a stream consisting essentially of n-butenes, a stream consisting essentially of heavier olefins, and an optional ethylene stream; and
optionally subjecting the stream consisting essentially of heavier olefins to hydrogenation so as to obtain renewably-sourced naphtha.

### Skeletal Isomerization of n-Butenes

In one aspect, the process of the invention involves isomerization of n-butenes obtained according to b)-(i) to obtain isobutene in accordance with step b)-(ii).

As the isomerization is an equilibrium reaction, the reaction mixture invariably contains unreacted n-butenes.

Expediently, step b)-(ii) comprise:
contacting the n-butenes with a skeletal isomerization catalyst in an isomerization zone to produce a mixture of n-butenes and isobutene;
recovering from the mixture a stream consisting essentially of n-butenes and a stream consisting essentially of isobutene; and
recycling the stream consisting essentially of n-butenes into the isomerization zone.

A suitable recovery scheme utilizes the reaction of isobutene with alkanol to produce alkyl tertiary butyl ether. The etherification reaction is selective with respect to isobutene, while n-butenes are unreactive in the reaction. The reaction therefore can be utilized as a method to separate n-butenes and isobutene.

Hence, isobutene may be recovered from the mixture of n-butenes and isobutene by the following steps:
(a) reacting the mixture of n-butenes and isobutene with isobutanol in the presence of an acidic ion exchange resin in an etherification unit to form a mixture of isobutyl tert-butyl ether (IBTBE) and unconverted n-butenes;
(b) distilling the reaction mixture in a first distillation unit to obtain a top product stream consisting essentially of n-butenes, and a bottom product comprising IBTBE;
(c) feeding the bottom product to a ether cleavage unit to decompose the IBTBE to obtain isobutene and isobutanol;
(d) distilling the mixture of isobutene and isobutanol produced in step (c) in a second distillation unit to obtain a top product stream consisting essentially of isobutene, and a bottom product comprising isobutanol; and
(e) recycling the bottom product of step (d) to step (a).

Skeletal isomerization generally requires acidic catalysts. Known skeletal isomerization catalysts include aluminas and halogenated aluminas, particularly For Cl-promoted aluminas.

Certain zeolites have been shown to be highly effective in skeletal isomerization of normal olefins. Such zeolites include those selected from the group consisting of zeolites having the framework structure of ZSM-22, ZSM-23, and ZSM-35.

Examples for high selectivity, high stability catalysts are chlorinated γ-Al₂O₃, ferrierite SAPO-11 (silico-alumino phosphate molecular sieve) and MeAPO-11 (Me=Co, Mn, Mg) (molecular sieve). A particularly preferred catalyst is ferrierite. The typical elemental composition of ferrierite zeolite is Na₂Mg₂[AlSi₃₀O₇₂]-18H₂O as, for example, disclosed in U.S. Pat. No. 6,323,384.

Spent catalysts can be regenerated by heating in an oxygen-containing gas, such as air, at temperatures ranging from about 200 °C to about 700 °C.

Skeletal isomerization of n-butenes to isobutene is an equilibrium controlled process where equilibrium conversion decreases with increasing temperature.

The skeletal isomerization is carried out by contacting the feed with the catalyst, using any suitable contacting techniques, at temperatures at which skeletal isomerization of the feed of n-butenes occurs. The feed is preferably maintained in the vapor phase during contacting. The reactor temperature is preferably in the range of about 300° to about 650 °C, more preferably about 400° to about 580 °C. The weight hourly space velocity (WHSV) is not narrowly critical but will generally be within the range of about 0.1 to about 40hr⁻¹, preferably from about 1 to about 20 hr⁻¹. Any convenient pressure can be used, with the lowest practical pressure preferred in order to minimize side reactions such as polymerization. Preferred pressures are within the range of about 0.1 to about 10 atmospheres, more preferably about 1 to about 4 atmospheres.

The equilibrium may not be achieved in the case of a single contact of the feed with the catalyst. However, in a particular variant of the process, the product stream leaving the catalyst bed can be divided up, and only one part is directly conveyed to the working-up process, while the other part is again conducted over the catalyst bed.

Several commercial processes for n-butene isomerization are known. In one embodiment the n-butene feedstock is vaporized, in general by heat exchange with reactor effluent, and further heated to reaction temperature. In the reactor, vapor reacts with up to 44% of n-butenes converted to isobutylene with greater than 86% selectivity. Typically, two reactors are cyclically operated: one in reaction mode, the other in regeneration mode. Reactor effluent is cooled, compressed and fractionated. Heavy ends are separated and removed as bottoms from the overhead isobutylene product.

Operating conditions, process and catalyst modifications are, for example, disclosed in U.S. Pat. Nos. 6,111,160 and 6,323,384. Typical operating conditions are: 340-360 °C reaction temperature, WHSV of 2H⁻¹, an olefin partial pressure of 1-2 bar, and a total pressure of 1-3 bar.

### Renewably Sourced C₄-Olefin Stream

The invention involves a renewably sourced C₄-olefin stream that is suitable for being subjected to olefin oligomerization. The C₄-olefin stream may comprise either n-butenes as the substantially only C₄-olefin, or isobutene as the substantially only C₄-olefin, or a mixture of n-butenes and isobutene. A C₄-olefin stream comprising a mixture of n-butenes and isobutene is obtained by blending n-butenes obtained according to (i) with isobutene obtained according to (ii) above.

The presence of isobutene in the C₄-olefin stream will result in branched higher olefins, which leads to branched alcohols. While highly branched alcohols have relatively little commercial value, a specific degree of branching is often desirable.

Technically, a distinction is often made between so-called “di-n-butenes”, i.e., isomeric Cg-olefins, which are prepared from mixtures of 1-butene and/or 2-butenes and so-called diisobutenes, which are obtained by dimerization of isobutene and which exhibit a higher degree of branching. Di-n-butenes are generally considerd to be better suited for the preparation of highly linear oxo-alcohols useful in preparing plasticizers than diisobutenes, since their degree of branching is much lower.

The isomeric distribution of isononanols or the degree of branching directly affects the properties of the plasticizer produced therefrom by esterification. Esterification with carboxylic acids, in particular phthalic acid, is well known. In industry, alcohol mixtures are esterified with phthalic acid or phthalic anhydride. The product, diisononyl phthalate (DINP), is used as a plasticizer. The structure of the alcohol, in turn, depends significantly on the structure of the parent olefin.

Effects of increased branching include adverse effects of increased viscosity, increased vapor pressure (which is associated with higher volatility), lower plasticization efficacy and lower thermal and light stability.

Conversely, high branching also leads to positive effects: better PVC miscibility, low migration, better hydrolysis resistance, low biodegradation (during the use phase) and high electrical resistance.

Depending on the intended use, trade-offs must be made. Therefore, if the plasticizing effect is of paramount importance, a plasticizer with minimal branching would be preferred. Conversely, if the goal is to produce a cable covered with PVC, a product with a somewhat higher branching would rather be chosen as the electrical insulating action would be better.

The invention allows for obtaining a desired degree of branching in accordance with the intended use, by adjusting the concentration of isobutene in the C₄-olefin stream. Also, the invention allows to mimic the butene composition in a butenes stream from a steam cracking unit. Once a recipe has been developed, it must now be ensured in the production process that a product with consistent properties is produced. If the isononanol is intended as a partial or full replacement for isononanol from fossil sources, the butene composition in the renewably sourced C₄-olefin stream can be made to resemble that of a butenes stream from a steam cracking unit.

In embodiments, the renewably sourced C₄-olefin stream comprises 0.2 to 8% by weight of isobutene, preferably 0.5 to 6% by weight of isobutene or 0.5 to 3% by weight of isobutene, relative to the total concentration of C₄-olefins in the C₄-olefin stream.

The renewably sourced C₄-olefin stream may be mixed with inert diluents, e.g., saturated hydrocarbons such as butanes, before being directed to the oligomerization unit. The olefin content of a butenes stream from a steam cracking unit is typically about 60% by weight, with the remainder being saturated hydrocarbons such as butanes. Generally, in the production of higher olefins, butanes are not removed from the butenes stream because a once through or low recycle oligomerization process is used. Instead, butanes are separated from the higher olefin product, which is a much easier and less costly separation.

The process of the invention provides a C₄-olefin stream with a high olefin content. This enables a high recycle, low conversion per pass oligomerization process. The high olefin content allows to maintain the olefin concentration in the feed at an acceptable level. The low conversion per pass process results in a higher selectivity to the more desirable alpha-olefins. Alpha-olefins are olefins that contain the carbon-carbon double bond between the first and second carbon.

### Oligomerization of Butenes

Step c) comprises an oligomerization reaction of the renewably-sourced C₄-olefin stream to produce dibutenes.

When butenes are subjected to oligomerization, olefins with eight carbon atoms (C₈-olefins, “dibutenes”), olefins with twelve carbon atoms (C₁₂-olefins, “tributenes”) and to a lesser extent, olefins containing more than twelve carbon atoms (C₁₂+-olefins) are obtained.

The separation of the obtained olefin oligomers from one another and from the unreacted C₄-olefins is well-known to the skilled person, and can be accomplished, e.g., via two-stage distillation.

The oligomerization of the C₄-olefin stream may be carried out by means of a fixed-bed catalyst, at superatmospheric pressure and at room temperature or elevated temperatures. The oligomerization is preferably carried out under supercritical conditions with respect to the starting material. For the oligomerization of C₄-olefins, reaction temperatures from 20 to 280 °C are preferred, more preferably above 160 ° C and in particular from 180 to 210 °C.

The reaction pressure is, in general, from 20 to 300 bar, in particular from 60 to 80 bar. In one embodiment, the oligomerization is carried out at a pressure from 60 to 300 bar, in particular from 60 to 80 bar, and at a temperature from 160 to 280 °C, in particular from 180 to 210 °C. In another embodiment, the oligomerization is carried out at a pressure from 10 to 30 bar, in particular from 15 to 25 bar, and at a temperature from 20 to 140 °C, in particular from 40 to 120 °C.

The catalyst used typically contains, as active components, after deduction of the loss on ignition following heating at 900 °C, from 10 to 70% by weight of nickel oxide, calculated as NiO, from 5 to 30% by weight of titanium dioxide or zirconium dioxide, from 0 to 20% by weight of alumina, from 20 to 40% by weight of silica and from 0.01 to 1 wt.-% of an alkali metal oxide, wherein the contents of the individual components in the catalyst add up to 100 wt.-%.

Further details with regard to the oligomerization process and the catalyst used are provided in WO 95/14647 A1 and WO 00/63151 A1 .

### Hydroformylation of Dibutenes

Hydroformylation reaction of the dibutenes with syngas yields isononanal.

Hydroformylation or the oxo process is an important large-scale industrial process for preparing aldehydes from olefins, carbon monoxide and hydrogen. These aldehydes can optionally be hydrogenated with hydrogen in the same operation or subsequently in a separate hydrogenation step, to produce the corresponding alcohols. In general, hydroformylation is carried out in the presence of catalysts which are homogeneously dissolved in the reaction medium.

Catalysts used are generally the carbonyl complexes of metals of transition group VIII, in particular Co, Rh, Ir, Pd, Pt or Ru, which may be unmodified or modified with, for example, amine-containing or phosphine-containing ligands. However, Co-catalysts are preferred for hydroformylation of dibutenes and tributenes. If alcohols having a very low degree of branching are desired, the hydroformylation reaction may preferably be carried out using unmodified cobalt catalysts. A summarizing account of the processes practiced on a large scale in industry is found in J. Falbe, “New Syntheses with Carbon Monoxide”, Springer Verlag 1980, p. 162 ff. Further details may be taken from U.S. Pat. No. 9,115,069, WO 2001/014297, WO 2021/197953 and U.S. Pat. No. 6,015,928.

Hydroformylation of dibutenes can be carried out at temperatures in the range of 120 °C to 240 °C, preferably 160 °C to 200 °C. The synthesis gas pressure is typically in the range of 150 to 400 bar, in particular from 250 to 350 bar. The molar ratio of hydrogen to carbon monoxide in the synthesis gas mixture used is preferably in the range from 70:30 to 50:50, in particular from 65:35 to 55:45.

In one embodiment, the hydroformylation reaction is conducted at a low pressure, e.g., a pressure in the range of 0.05 to 50 MPa (absolute), and preferably in the range of about 0.1 MPa to 30 MPa, most preferably at a pressure below 5 MPa. Desirably, the partial pressure of carbon monoxide is not greater than 50% of the total pressure.

The proportions of carbon monoxide, hydrogen, and dibutenes in the hydroformylation reaction medium can be selected within a wide range. In some embodiments, based on the total amount of CO, hydrogen, and dibutenes, CO is from about 1 to 50 mol-%, preferably about 1 to 35 mol-%; H₂ is from about 1 to 98 mol-%, preferably about 10 to 90 mol-%; and dibutenes are from about 0.1 to 35 mol-%, preferably about 1 to 35 mol-%.

The hydroformylation reaction preferably takes place in the presence of both liquid and gas phases. The reactants generally are in the gas phase. The catalyst typically is in the liquid phase. Because the reactants are gaseous compounds, a high contact surface area between the gas and liquid phases is desirable to enhance good mass transfer. A high contact surface area between the catalyst solution and the gas phase may be provided in any suitable manner. In a batch process, the batch contents are thoroughly mixed during the course of the reaction. In a continuous operation the reactor feed gas can be contacted with the catalyst solution in, for example, a continuous-flow stirred autoclave where the gas is introduced and dispersed at the bottom of the vessel, preferably through a perforated inlet (e.g., a sparger). High contact between the catalyst and the gas feed may also be provided by dispersing the solution of the Rh catalyst on a high surface area support, a technique well known in the art as supported liquid phase catalysis, or providing the Rh as part of a permeable gel.

The reaction may be conducted either in a batch mode or, preferably, on a continuous basis. One or more reactors may be used in continuous modes to carry out the reaction in one or more stages.

### Production of Syngas

The syngas used in the hydroformylation reaction of step B ) may be obtained by:
subjecting a gasifier feed stream comprising a renewably sourced material to gasification in a gasifier to obtain a gasifier effluent;
recovering syngas from the gasifier effluent.

Preferably, the feedstock for the gasifier is selected from the group comprising biomass, municipal solid waste (MSW), shredder residues such as car shredder residues, textiles, plastic waste, packaging waste, and mixtures thereof. Such feedstocks can also be mixed with fossil feedstocks such as coal, oil, and natural gas. The amount of fossil feedstocks is typically not more than 10 wt.-%, preferably not more than 5 wt.-%.

The term “biomass” includes but is not limited to wood, wood pellets, wood chips, straw, lignocellulosic biomass, energy crops, algae, biobased-oils, biobased-fats, and mixtures thereof.

The term “waste” comprises fossil-based waste, biogenic waste, and mixtures thereof. Examples for waste suitable as a feedstock are agricultural/farming residues such as wood processing residues, waste wood, logging residues, switch grass, discarded seed corn, corn stover and other crop residues, municipal solid waste (MSW), textiles, industrial waste, sewage sludge, plastic waste, packaging waste, shredder residues such as car shredder residues and mixtures thereof.

Optionally, the feedstock is pre-treated before entering the gasifier. A suitable pre-treatment method or combination of pre-treatment methods in a pre-treatment unit should provide a sufficiently homogeneous carbon-based feedstock to the gasification reaction and likewise enable the continuous production of syngas by gasification of a feedstock.

A pre-treatment method or a combination of more than one pre-treatment methods in a pre-treatment unit preferably results in a homogenization of the physical and/or chemical properties of the first feedstock and/or the second feedstock and/or the requirement(s) for a specific type of gasifier and/or the requirements for the optional at least one further chemical production unit for producing a chemical compound or mixture of chemical compounds.

The pre-treatment method for the first feedstock and/or the second feedstock is preferably selected from the group comprising drying, comminution, classification, sorting, agglomeration, thermochemical methods, and biological methods.

Suitable gasifiers comprise counter-current fixed bed reactors, co-current-fixed bed reactors, bubbling fluidized bed reactors, circulation fluidized bed reactors, and downdraft or updraft entrained flow reactors. The selection of size and reactor type depends on several parameters, including the composition of the (carbonaceous) feedstock, demand of products, moisture content and availability of the (carbonaceous) feedstock. Preferably, the gasifier is an “oxygen blown” gasifier, i.e., oxygen is preferably used as the oxidant in suitable gasifiers listed above.

The gasification reaction in a gasifier is typically carried out at a temperature of greater than 700 °C in the presence of a sub-stoichiometric amount of an oxidant such as oxygen, air, steam, supercritical water, CO₂, or a mixture of the aforementioned. Preferably the gasification is carried out at a temperature in the range of greater than 700 to 1500 °C, more preferably, 850 to 1400 °C, even more preferably 1100 to 1500 °C. Typically, the gasification is conducted at an absolute pressure of greater than 1 bar. Preferably it is conducted at an absolute pressure in the range from 2 to 80 bar, more preferably 2 to 50 bar. Oxygen is the most common oxidant used for gasification because of its easy availability and low cost. The H₂:CO ratio depends on the composition of the feedstock and the amount of steam used in the gasification. The H₂:CO ratio as required for the hydroformylation can for instance be adjusted by choosing an appropriate amount of steam in the gasification.

Another possibility to adjust the H₂:CO ratio is to separate CO from the syngas. CO can be separated from the syngas in a syngas separation unit which is downstream of and fluidly connected to the syngas producing unit comprising at least one gasifier. CO can be separated from syngas by cryogenic separation methods, commonly referred to as a “cold box” which makes use of the different boiling points of CO and H₂. H₂ can be separated using H₂-selective membranes thorough which H₂ permeates and is thereby separated from a syngas stream. It is also possible to fully separate CO and H₂ via cryogenic separation. The resulting CO and H₂ can be used to create a syngas having the desired ratio.

When steam acts as oxidant, the syngas has a higher molar ratio H₂:CO than when air is used as oxidant. For example, a typical molar ratio of “air:combined feedstock ” ranges from 0.3 to less than 1.

The conversion of a feedstock in the gasifier produces a syngas which consists primarily of H₂, CO, CO₂, methane, other hydrocarbons, and impurities. Said syngas has a dedicated molar ratio H₂:CO when leaving the gasifier which ranges from about 0.1:1 to about 3:1 and depends on the type of solid and/or liquid feedstocks used, the oxidant and other reaction conditions applied such as temperature and/or residence time of the reactants in the gasifier.

Typical impurities in the raw syngas obtained from the gasification reaction in a gasifier comprise chlorides, sulfur-containing organic compounds such as sulfur dioxide, trace heavy metals (e.g., as respective salts) and particulate residues. Various chemical and/or physical methods for removal of such impurities from said raw syngas such as filtration, scrubbing, hydrotreatment and ab-/adsorption are known and can be chosen and adapted according to the type and respective concentration of the impurities in said raw syngas and the tolerance to such impurities in the successive process steps. Some selected methods for removal of impurities from said raw syngas will be discussed in more detail. One or more of said methods can also be implemented into the at least one syngas purification unit of the syngas producing unit comprising at least one gasifier. However, this selection of methods is not limiting the scope of the present invention.

Bulk particulate impurities can be removed from the raw syngas by a cyclone and/or filters, fine particles, and chlorides by wet scrubbing, trace heavy metals, catalytic hydrolysis for converting sulfur-containing organic compounds to H₂S and acid gas removal for extracting sulfur-containing gases such as H₂S. Bulky and fine particles in the syngas may also be removed with a quench in a soot water washing unit.

A gasification reaction usually results in further reaction products such as solid and/or highly viscous carbonaceous residues (e.g., char and/or tar) which can be further treated in separate steps not relevant for the systems and methods according to the present invention.

### Hydrogenation of Isononanal

Hydrogenation of the isononanal yields isononanol. The hydrogenation of the isononanal to isononanol is a well-known reaction and can be conducted by any suitable known process.

In one embodiment, the hydrogenation is carried out with hydrogen in the liquid or gas phase in the presence of a hydrogenation catalyst. Homogeneous or heterogeneous catalysts can be used. Copper catalysts have proved to be the most suitable. Typically, the reaction is carried out in the liquid phase on fixed-bed catalysts at 50 to 250 °C and pressures of up to 300 bar. Hydrogenation in the gas phase is preferably carried out continuously. The desired isononanol fraction in the reaction discharge obtained during the hydrogenation can be separated off by fractional distillation from the C₈ hydrocarbons and higher-boiling products. Further details can be taken from Ullmann's Encyclopedia of Industrial Chemistry, 5^{th} edition, vol. A1, 1984.

### Producing a Diester of Adipic Acid or Phthalic Acid

The present invention further provides a process for producing a diester of adipic acid or phthalic acid, comprising subjecting adipic acid, phthalic acid, or a derivative thereof to an esterification reaction with an isononanol obtained according to any one of the preceding claims.

Diesters obtained from isononanol and adipic acid or phthalic acid are generally known and are important, for example, as plasticizers for preparing thermoplastic molding compositions.

Adipic acid and phthalic acid can be esterified with isononanol in a conventional manner to produce the desired diester.

The phthalates according to the invention are preferably prepared using phthalic anhydride. Isononanol obtained as described above is preferably reacted in excess with the adipic acid, phthalic acid, or derivative thereof, in particular in a molar excess of 5 to 30%, preferably in the presence of an acylation catalyst, such as a dialkyl titanate, e.g. isopropyl butyl titanate, or of an acid, such as methanesulfonic acid or sulfuric acid.

The reaction with the adipic acid, phthalic acid, or derivative thereof generally takes place at temperatures of from 150 to 300 °C, preferably from 200 to 250 °C. In an appropriate embodiment, an inert gas, such as nitrogen, is bubbled into the reaction mixture during the reaction and the water formed in the reaction is removed progressively from the reaction mixture by the inert gas stream. Once the reaction has ended, diesters of adipic or phthalic acid are isolated from the reaction mixture, by, for example, distilling off excess isononanol in vacuo, neutralizing the crude diester with an aqueous alkali, such as aqueous sodium hydroxide, forming a two-phase mixture, separating off the aqueous phase, and washing the organic phase. For further purification, the neutralized and washed diester is preferably stripped using steam at elevated temperature in vacuo. The purified diester may then be dried at elevated temperature in vacuo by passing a nitrogen stream through the mixture, and, if desired, further purified by being brought into contact with an adsorbant, such as activated carbon or bleaching earth.

The diisononyl adipates according to the invention prepared in this way have a density of from 0.900 to 0.940 g/cm³, preferably from 0.910 to 0.930 g/cm³, in particular from 0.918 to 0.922 g/cm³, a viscosity of from 15.0 to 25.0 mPa·s, preferably from 16.0 to 22.0 mPa·s, particularly preferably from 17.0 to 20.0 mPa·s, and a refractive index nₚ²⁰ of from 1.445 to 1.455, preferably from 1.447 to 1.453, particularly preferably from 1.448 to 1.452.

The diisononyl phthalates according to the invention prepared in this way generally have a density of from 0.950 to 0.990 g/cm³, preferably from 0.960 to 0.980 g/cm³, in particular from 0.967 to 0.973 g/cm³, a viscosity of from 60.0 to 110.0 mPas, preferably from 63.0 to 80.0 mPa·s, in particular from 65.0 to 75.0 mPas, and a refractive index np 20 of from 1.470 to 1.495, preferably from 1.476 to 1.490, in particular from 1.480 to 1.486.

1.-7. (canceled)

8. A process for the manufacture of isononanol that is a mixture of isomeric nonyl alcohols, said process comprising the steps of:
a) subjecting a feedstock comprising a renewably-sourced ethanol to dehydration to produce a renewably-sourced ethylene stream;
b) subjecting the renewably-sourced ethylene stream to an olefin-interconversion to obtain a renewably-sourced C4-olefin stream, the renewably-sourced C4-olefin stream comprising n-butenes, isobutene or a mixture of n-butenes and isobutene; the olefin-interconversion comprising (i) and, where required, (ii), or (ii) and (iii):
(i) ethylene dimerization to obtain n-butenes; comprising
contacting the renewably-sourced ethylene stream with a dimerization catalyst in a dimerization zone;
operating said dimerization zone at conditions effective to produce an effluent consisting essentially of n-butenes, a stream consisting essentially of heavier olefins, and optionally an unconverted ethylene stream; and
fractioning the effluent to recover a stream consisting essentially of n-butenes, a stream consisting essentially of heavier olefins, and an optional ethylene stream;
(ii) isomerization of n-butenes obtained according to (i) to obtain isobutene;
(iii) blending n-butenes obtained according to (i) with isobutene obtained according to (ii); and
c) subjecting the renewably-sourced C4-olefin stream to a sequence of chemical conversions to obtain isononanol, comprising:
α) oligomerizing the renewably-sourced C4-olefin olefin stream to produce dibutenes;
β) subjecting the dibutenes to a hydroformylation reaction with syngas to obtain isononanal; wherein the syngas is obtained by:
subjecting a gasifier feed stream comprising a renewably-sourced material to gasification in a gasifier to obtain a gasifier effluent; and
recovering syngas from the gasifier effluent; and
γ) hydrogenating the isononanal to produce isononanol.

9. The process according to claim 8, comprising
blending the renewably-sourced ethylene with complementary ethylene prior to step b), the complementary ethylene not being obtained from renewably-sourced ethanol in accordance with step a); and/or p1 blending the renewably-sourced n-butenes with complementary n-butenes prior to step b-(ii), b-(iii) or c), the complementary n-butenes not being obtained from renewably-sourced n-butenes in accordance with steps a) and b-(i); and/or p1 blending the renewably-sourced isobutene with complementary isobutene prior to step b-(iii) or c), the complementary isobutene not being obtained from renewably-sourced n-butenes in accordance with steps a), b-(i) and b-(ii).

10. The process according to claim 8, wherein step b)-(ii) comprises:
subjecting the n-butenes to skeletal isomerization to produce a mixture of n-butenes and isobutene;
recovering from the mixture a stream consisting essentially of n-butenes and a stream consisting essentially of isobutene; and
recycling the stream consisting essentially of n-butenes into the skeletal isomerization.

11. The process for producing a diester of adipic acid or phthalic acid, comprising obtaining isononanol in accordance with the process according to claim 8, and subjecting adipic acid, phthalic acid, or a derivative thereof to an esterification reaction with the obtained isononanol.

12. The process for enhancing the environmental sustainability of isononanol that is a mixture of isomeric nonyl alcohols, by blending or replacing a fossil-derived C₄-olefin stream comprising n-butenes, isobutene or a mixture of n-butenes and isobutene with a renewably-sourced C₄-olefin stream of the same composition to obtain a sustainability-enhanced C₄-olefin stream and subjecting the sustainability-enhanced C₄-olefin stream to a sequence of chemical conversions to obtain isononanol, wherein the renewably-sourced C₄-olefin stream is obtained by p1 a) subjecting a feedstock comprising a renewably-sourced ethanol to dehydration to produce the renewably-sourced ethylene stream; and
b) subjecting the renewably-sourced ethylene stream to an olefin-interconversion, to obtain the renewably-sourced C4-olefin stream; the olefin-interconversion comprising (i) and optionally (ii), or (ii) and (iii):
(i) ethylene-dimerization to obtain n-butenes; comprising:
contacting the renewably-sourced ethylene stream with a dimerization catalyst in a dimerization zone;
operating said dimerization zone at conditions effective to produce an effluent consisting essentially of n-butenes, a stream consisting essentially of heavier olefins, and optionally an unconverted ethylene stream; and
fractioning the effluent to recover a stream consisting essentially of n-butenes, a stream consisting essentially of heavier olefins, and an optional ethylene stream;
(ii) isomerization of n-butenes obtained according to (i) to obtain isobutene;
(iii) blending n-butenes obtained according to (i) with isobutene obtained according to (ii);
the sequence of chemical conversions comprising:
α) oligomerizing the n the renewably-sourced C4-olefin olefin stream to produce dibutenes;
β) subjecting the dibutenes to a hydroformylation reaction with syngas to obtain isononanal; wherein the syngas is obtained by:
subjecting a gasifier feed stream comprising a renewably-sourced material to gasification in a gasifier to obtain a gasifier effluent; and
recovering syngas from the gasifier effluent; and
γ) hydrogenating the isononanal to produce isononanol.
